(19) United States
(12) Patent Application Publication
(10) Pub. No.: US 2026/0334985 A1
(43) Pub. Date: Oct. 29, 2026

(54) BONE SCAFFOLD WITH SONOLUCENT WINDOW

(71) Applicants: BROWN UNIVERSITY, Providence, RI (US); GENERAL ELECTRIC COMPANY, Schenectady, NY (US); RHODE ISLAND HOSPITAL, Providence, RI (US)

(72) Inventors: David BORTON, Providence, RI (US); Steven J. DUCLOS, Schenectady, NY (US); Jared FRIDLEY, Providence, RI (US); Guatam PARTHASARATHY, Schenectady, NY (US); Kirk D. WALLACE, Schenectady, NY (US)

(21) Appl. No.: 19/104,034

(22) PCT Filed: Aug. 16, 2022
(86) PCT No.: PCT/US2022/040397

§ 371 Date: Feb. 14, 2025

(51) Int. Cl. A61B 17/70; A61B 8/06; A61B 17/00

(52) U.S. Cl. CPC A61B 17/7049; A61B 8/06; A61B 2017/00924; A61B 2017/00982

(57) ABSTRACT

An implantable medical device configured to receive ultrasound energy is provided. The implantable device includes a sonolucent window that is fabricated from a sonolucent material and that is configured to allow US energy to pass therethrough. The medical device can be a bone scaffold. The medical devices can be used for imaging, monitoring, or therapeutic/treatment purposes.

### TECHNICAL FIELD

The present disclosure relates to a bone scaffold with an integrated sonolucent window that allows ultrasound energy to pass through the window.

### BACKGROUND

Spinal cord injury (SCI) is a debilitating condition that disrupts signal propagation and autonomic regulation and has long-term health implications in addition to a heavy financial burden on the individual and society. Following a traumatic SCI, the current standard of care focuses on decompression of the spinal cord injury site, realignment and stabilization of the spinal column, and hemodynamic maintenance to minimize secondary injury. Ensuring biomechanical stability concomitantly with adequate spinal perfusion and oxygenation is critical in preventing secondary injury to the spinal cord as posttraumatic ischemia has been linked to axonal dysfunction following acute SCI. Unfortunately, limited methods exist to monitor posttraumatic ischemia and adequately visualize the longitudinal progression of the injured spinal cord in the post-operative setting. Functional and structural Magnetic Resonance Imaging (MRI) is expensive and time-consuming. Further, some patients cannot tolerate MRIs due to claustrophobia or anxiety. Spinal hardware artifact can also limit the view of the spinal cord on MRI, making evaluation of the spinal cord challenging. Patients with certain implanted metal devices or other ferromagnetic objects in their body (e.g. a pacemaker, metal mesh, shrapnel, etc.) cannot obtain MRIs. The alternative imaging modality for a patient unable to undergo MRIs is a myelogram followed by a spinal computerized tomography (CT) scan. However, this imaging modality involves injecting contrast dye in the patient's subdural space, which carries a risk of infection, CSF leak, and potential neurologic injury. Further, CT is useful primarily for assessing bony injury, and provides little information on spinal cord integrity, other than indirectly by assessing the size of the spinal canal and the presence of any canal occupying bony fragment or hematoma. Indirect measures of mean arterial pressure (MAP) can be variable and unreliable. Further, none of the modalities are performed longitudinally.

Ultrasound (US) imaging is routinely used intra-operatively in the spine to identify spinal cord anatomy and associated pathology, such as during resection of intramedullary tumors. However, the use of US as a spinal cord imaging modality in the post-operative and longitudinal setting has yet to be developed. This is mainly due to the acoustic impendence mismatch between the bony spine and the soft tissue of the spinal cord, preventing clear imaging of spinal cord structures.

As such, the development of an alternative device and method to capture the structure and hemodynamics of the spinal cord that is versatile, reliable, non-invasive, and not significantly affected by spinal hardware artifact is needed.

### SUMMARY

### BRIEF DESCRIPTION OF THE DRAWINGS

FIG. 1 is a perspective view of a medical device according to an aspect of the present disclosure.

FIG. 2 is a top view of a medical device inserted in the vertebral column according to an aspect of the present disclosure.

FIG. 3 is a top view of the medical device of FIG. 2 illustrating the medical device in a rotated position.

FIG. 4 is a top view of a medical device inserted in the vertebral column according to an aspect of the present disclosure.

FIG. 5 is a top view of a medical device inserted in the vertebral column according to an aspect of the present disclosure.

FIG. 6 is a top view of a medical device inserted in the vertebral column according to an aspect of the present disclosure.

FIG. 7 is a flow chart depicting steps of a method of using a medical device according to an aspect of the present disclosure.

FIG. 8 is a flow chart depicting steps of a method of using a medical device according to an aspect of the present disclosure.

FIG. 9 is a flow chart depicting steps of a method of using a medical device according to an aspect of the present disclosure.

FIG. 10 is a flow chart depicting steps of a method of using a medical device according to an aspect of the present disclosure.

### DETAILED DESCRIPTION

As used herein with respect to a described element, the terms “a,” “an,” and “the” include at least one or more of the described elements including combinations thereof unless otherwise indicated. Further, the terms “or” and “and” refer to “and/or” and combinations thereof unless otherwise indicated. By “substantially” is meant that the shape or configuration of the described element need not have the mathematically exact described shape or configuration of the described element but can have a shape or configuration that is recognizable by one skilled in the art as generally or approximately having the described shape or configuration of the described element. A “patient” as described herein includes a mammal, such as a human being. The term “top,” “bottom,” “upper,” “lower,” “above,” and “below” refer to the position or orientation of the components as depicted in the drawings. The terms “first,” “second,” etc. are used to distinguish one element from another and not used in a quantitative sense unless indicated otherwise. The term “plurality” includes two or more of the described components. In addition, when an element is referred to as being “on,” “attached” to, “connected” to, “coupled” with, “contacting,” in “communication” with, “extending” from etc., another element, it can be directly on, attached to, connected to, coupled with, contacting, in communication with, or extending from the other element or intervening elements may also be present. In contrast, when an element is referred to as being, for example, “directly on,” “directly attached” to, “directly connected” to, “directly coupled” with, “directly contacting,” in “direct communication” with, or “directly extending” from another element, there are no intervening elements present. An element that is disposed “adjacent” to another element may have portions that overlap or underlie the adjacent element. By “monolithic” is meant an integral component that is a single piece or multiple pieces affixed during manufacturing and the described components are otherwise not separable using a normal amount of force without damaging the integrity (i.e., tearing) of either of the components. A normal amount of force is the amount of force a user would use to remove a component meant to be separated from another component without damaging either component. All devices described herein are for medical purposes and are therefore sterile.

The present disclosure relates to medical devices that are configured to receive US energy for diagnostic, monitoring, and/or therapeutic purposes. Although described below primarily in the context of longitudinally visualizing and monitoring spinal cord structure, spinal perfusion, and hemodynamics of a spinal lesion and spinal cord regions adjacent to the spinal lesion in patient with SCI, the disclosed devices and methods can be used in any medical context where US energy can be delivered to a patient including, for example, myelopathy.

In the context of spinal lesions. such medical devices can allow clinicians and researchers to use trans-spinal ultrasound (tsUS) to observe changes immediately following SCI and over longer, chronic time spans. Visualization of the spinal lesion can provide an opportunity to directly study the effects of clinical interventions and treatments of the spinal lesion and spinal cord. Such devices can assist in the prognosis of spinal cord injury to aid clinicians in determining the likely progression of the spinal cord injury. Additionally, such medical devices can provide quantitative and qualitative information about the state of the spinal cord and spinal lesion post-injury, leading to the discovery of biomarkers that may aid in determining the status of the spinal cord following injury.

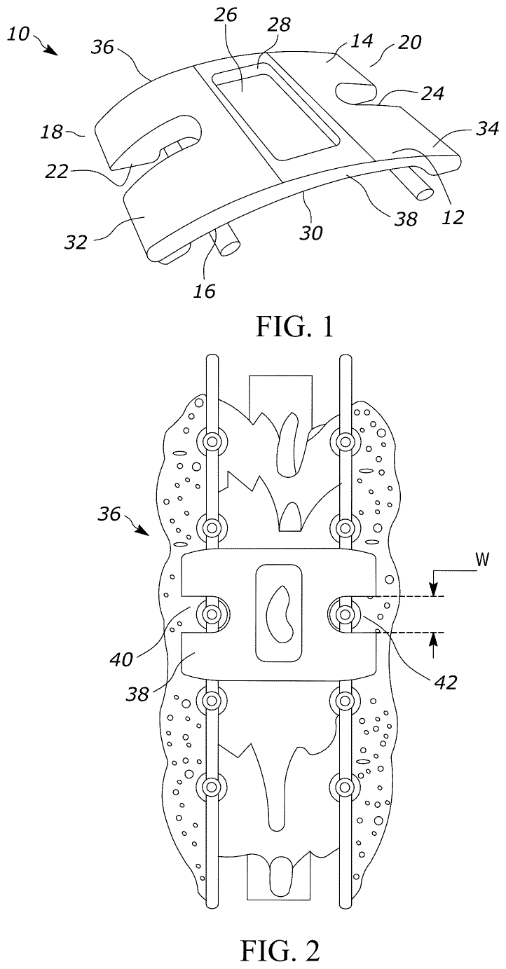

In an aspect and with reference to FIG. 1, medical device 10 is configured to receive US energy. Medical device 10 can comprise device body 12 having outer surface 14, inner surface 16, first side 18, and second side 20. In certain aspects, a first notch 22 can be located on first side 18 and can be sized and configured to receive a bone fastener. Non-limiting examples of bone fasteners includes screws, sutures, threads, mechanical clamps, etc. A second notch 24 can be located on second side 20 and can be sized and configured to receive a bone fastener. In embodiments with notches, the notches serve to “clear” the fasteners or other instrumentation that may be implanted at the target site (e.g. rods). In certain aspects, the inner surface of the scaffold can comprise an adhesive to adhere the device to the target site. Although FIG. 1 illustrates only two notches defined by the device body, the device body can include any suitable number of notches to accommodate the relevant stabilization instrumentation. Medical device body can also include different port holes 54 for delivery of other therapies such as, for example drug delivery and electrical stimulation, described in more detail below.

Sonolucent window 26 can be disposed in device body 12 and can be sized, configured, and fabricated from a sonolucent material to allow US energy to pass therethrough. In certain aspects, the sonolucent material has a mass density and an elastic modulus that corresponds to an intrinsic longitudinal sound speed value close to a nominal soft tissue sound speed of 1540 m/s and an acoustic impedance value close to a nominal soft tissue value of 1.5 MRayls. In certain aspects, the sonolucent material is homogenous and void free.

As shown in the figures, the sonolucent window can be disposed between the outer surface and the inner surface of the medical device body. Although the figures illustrate the sonolucent window between top face 28 and bottom face 30, the sonolucent window can be disposed between other faces of the medical device such as between the inner and outer surfaces of respective first side face 22, second side face 24, top face 36 and/or bottom face 38. In certain aspects, the sonolucent window is approximately 2 inches thick. The window is large enough to prevent bony bridging and overgrowth across the medical device. In the case of the implantation site of the medical device being the spinal cord, the sonolucent material of the window may serve many purposes including, for example, protecting the spinal cord when placing an ultrasound imaging probe above the site for imaging protocols (e.g. perioperatively before the incision site is closed); creating an acoustic propagation path that is homogenous (e.g. minimal scattering and contributions to image clutter artifacts) and well-matched to intrinsic soft tissue longitudinal speed of sound (minimal diffraction artifacts) and specific acoustic impedance (minimal reflection artifacts) properties; providing a physical standoff that is configured to be positioned in the spinal cord region of interest within approximately 12 to approximately 28 mm optimal depth of field; creating optical transparency to aid in clinician lateral placement over the lesion; and facilitating CT and MR imaging compatibility to minimize the impact on long-term patient care.

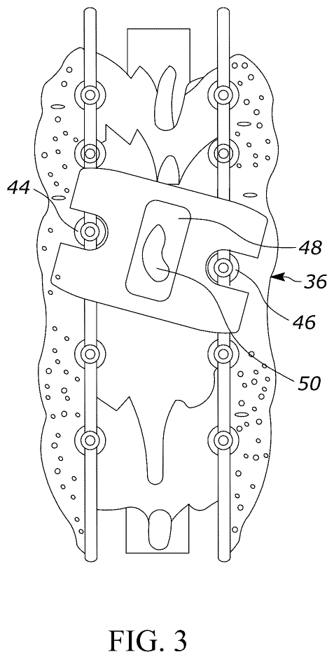

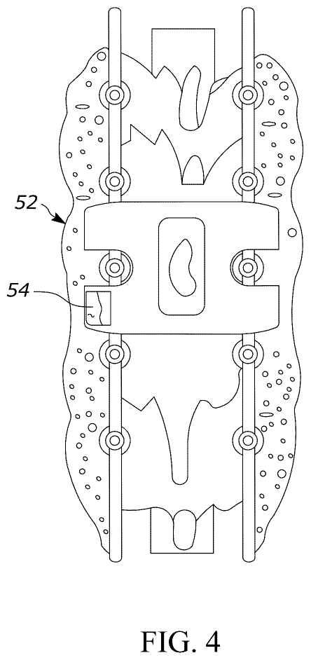

FIGS. 2-6 illustrate a medical device that is a bone scaffold 36 with a scaffold body 38 and optional opposing first and second notches 40 and 42 that are sized and configured to receive respective bone fasteners (e.g. facet fasteners, pedicle fasteners, etc.) 44 and 46. FIGS. 2-6 illustrate the bone scaffold implanted over a spinal cord lesion site 50 (e.g. during decompression surgery) in conjunction with standard of care stabilization fasteners and rods. Referring to FIG. 3, bone scaffold 36 can be rotated to any suitable angle in order for the sonolucent window to be properly positioned over the internal body site (e. g spinal lesion site 50) such that US can pass therethrough to the internal body site.

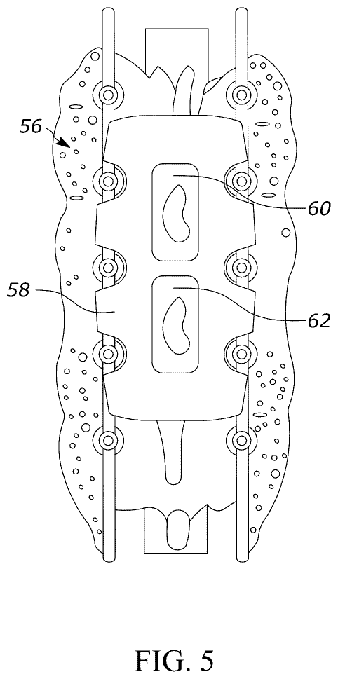

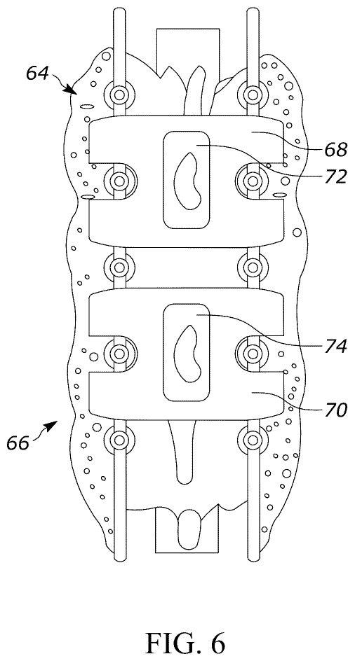

Although FIGS. 2-4 depict a single medical device body (in this instance a single bone scaffold body), the medical device can include a plurality of medical device bodies, a plurality of sonolucent windows, or any suitable and reasonable combination thereof. FIG. 5 depicts bone scaffold 56 comprising a monolithic bone scaffold body 58 comprising two sonolucent windows 60 and 62 that are vertically adjacent to one another (extending along the sagittal plane of the patient) and FIG. 6 depicts two separate and distinct bone scaffolds 64 and 66 with separate respective bone scaffold bodies 68 and 70 and separate respective sonolucent windows 72 and 74.

A bone scaffold as described herein includes an osseoinductive and an osseoconductive supporting scaffold body that can promote structural integration with the vertebrae and support mechanical stabilization post-SCI. The scaffold can, for example, be loading bearing, promote bone growth, and promote vascularization. The implantable device body can include, for example, shaved lamina from the patient, bone morphogenic proteins BMPs) or other substances to stimulate bone growth; vascular endothelial growth factors (VEGF) or other substances to promote neovascularization; fibronectin (FN) or other substances that promote tissue repair; and suitable combinations thereof.

The sonolucent window embedded in the bone scaffold body can facilitate non-invasive application of trans-spinal ultrasound (tsUS) and monitoring of a spinal lesion for an extended period of time (e.g. the lifetime of the patient). As such, bone scaffolds as described herein can provide non-invasive, longitudinal trans-spinal imaging of the spinal cord. The bone scaffold can facilitate safe and reproducible ultrasound imaging of the progression of spinal cord healing post-SCI decompression surgery; accelerate the development of bony tissue disrupted by the SCI; and facilitate the observation of interventional impact on spinal hemodynamics.

The bone scaffold body can be fabricated, for example, from a ceramic material, a polymeric material, a composite material, or suitable combinations thereof. The bone scaffold body can also have open-cell porosity for the delivery of therapeutic agents. The sonolucent window is fabricated from a sonolucent material. Non-limiting examples of sonolucent materials include thermoplastic materials that preferably have favorable properties that are well matched to soft tissue applications including, for example, polymethyl-pentene, polymethyl methacrylate, polytetrafluoroethylene (PTFE), polyether-ether-ketone (PEEK), and suitable combinations thereof. The sonolucent window can be embedded into the bone scaffold body via an adhesive (e.g. cyanoacrylate adhesive, epoxy), a snap-fit design, other fixation mechanisms, and combinations thereof. The sonolucent window can be integrated into the bone scaffold body such that it is not meant to be separated from the bone scaffold body or can be removably attached to the bone scaffold body.

The present disclosure also provided different methods of using medical devices as described herein. Again, the below disclosure will be described primarily with respect to a bone scaffold, but such methods can apply to other medical devices and other internal body sites where US energy can be delivered to a patient including the head (e.g. cranium), spine, or limbs of a patient, for example. Referring to FIG. 7, in an aspect, a method of imaging an internal body site of a patient 100 is provided. Method 100 can include positioning a medical device with a sonolucent window at the internal body site such that the internal body site is accessible to ultrasound energy through the sonolucent window 102. Method 100 can further include applying, through the sonolucent window, ultrasound energy to the internal body site to produce an image of the internal body site 104. In certain instances, the medical device is a bone scaffold and the internal body site is the spinal cord (e.g. the cervical, thoracic, lumbar, sacral spinal regions, or combinations thereof). The internal body site can comprise a spinal cord lesion from a SCI, the region adjacent to the spinal cord lesion, or both. Such methods can provide details regarding the spinal cord structure (including e.g. grey and white matter tracts), perfusion to the spinal cord lesion site, or both in a patient who has suffered a SCI. For example, such methods can detect different between an intact spinal cord and a spinal cord that has been acutely injured. In certain aspects, a laminectomy is performed to remove bone from the lumbar spine and the bone scaffold is aligned with the remaining bone to serve as a platform for osseointegration and tissue growth. The bone scaffold can be stabilized in the spine with facet joints, for example. After implantation of the bone scaffold, the patient can undergo ultrasound imaging to acquire spinal cord images, including distinctions/boundaries between grey and white matter, over time. For example, the axial structure of the lumbosacral spinal cord can be assessed using an ultrasound probe that is moved along the longitudinal axis of the spinal cord to determine any structural changes or differences in grey and white matter composition at different spinal segments. The internal body site can include other regions as well and the ultrasound imaging can be used to image a vascular malformation, a tumor, a hemotoma, contusion or other formation in the body.

Referring to FIG. 8, in another aspect a method of monitoring a medical condition or characteristic thereof in a patient 200 is provided that comprises positioning a medical device with a sonolucent window at an internal body site associated with the medical condition such that the internal body site is accessible to ultrasound energy through the sonolucent window 202. Method 200 further comprises applying, through the sonolucent window, US energy to the internal body site 204 and measuring a physiological parameter associated with the medical condition based on the application of ultrasound energy 206. Method 200 can further include monitoring the medical condition or characteristic thereof based on the measurement of the physiological parameter 208. In certain instances, the medical device is a bone scaffold and the internal body site is the spinal cord (e.g. the cervical, thoracic, lumbar, sacral spinal regions, or combinations thereof). The internal body site can comprise a spinal cord lesion from a SCI, the region adjacent to the spinal cord lesion, or both. Doppler US imaging can be used to quantify blood flow values along the spinal cord blood vessels to the spinal cord lesion and/or a region adjacent to the spinal cord lesion to determine a baseline perfusion value. This baseline value can be compared to subsequent blood flow values quantified using Doppler US imaging to determined changes in perfusion to the spinal cord lesion and/or a region adjacent to the spinal cord region over time, and the ability to modulate spinal perfusion according to non-limiting therapies described below. Spatial differences in hemodynamics at the internal body site can also be determined using ultrasound. In some instances, the internal body site is a site of trauma and the US can be used to monitor the progression of the trauma such as a hemotoma, contusion, or other injury/damage to tissue. Other physiological parameters can be monitored including intracranial pressure.

Referring to FIG. 9, in certain aspects a method of improving a medical condition is provided. For example, a method of improving a medical condition or characteristic thereof in a patient 300 can comprise positioning a medical device with a sonolucent window at an internal body site associated with the medical condition such that the internal body site is accessible to ultrasound energy through the sonolucent window 302. Method 300 can comprise applying, through the sonolucent window, ultrasound energy to the internal body site 304. Method 300 includes improving the medical condition via application of the ultrasound energy 306. The ultrasound energy can have a neuromodulatory effect on the internal body site. In certain aspects, the US is high-frequency ultrasound (HIFU) (e.g. approximately 650 Khz) to thermally ablate a target body site for medical conditions such as essential tremor, Parkinson's disease, epilepsy, neuropathic pain, a tumor (e.g. a bony tumor in the spine or limbs) obsessive compulsive disorder, or dystonia, for example. Alternatively, low frequency US (LIFU) (e.g. approximately 220 Khz) can be applied to open the blood brain barrier (e.g. in conjunction with the delivery of microbubbles) to delivery therapeutic agents to a target site for brain tumors, neurodegenerative disorder, and stroke for example. LIFU could also be applied for purposes of reversible neuromodulation. A neuromodulation can affect the neuronal activity, chemistry, and/or metabolism of the nerves/neural tissue of the internal body side. The effect can include an increase, decrease, or even a change in a pattern of neuronal activity and can mask, alter, override, or restore neuronal activity.

In certain instances, the medical condition or characteristic thereof is back pain and the internal body site is the spine. The medical device can be positioned at a certain spinal level associated with the back pain. The medical device can also contain therapeutic agents that can be released at the internal body site to further alleviate the pain or can contain a port sized and configured to receive a drug delivery needle to locally inject a therapeutic agent (e.g. an opioid) for outside the patient's body. Other therapeutic agents can be released (e.g. fast release, slow release, controlled release, sustained release) such as antibiotics to treat an infection, chemotherapeutic agents to treat medullary tumors or other cancers; anti-inflammatory agents to treat inflammation, and other therapeutic agents depending on the medical condition or characteristic. Non-limiting examples of other medical conditions that can be improved by method described herein including vascular, infectious, neoplastic, degenerative, inflammatory, congenital, autoimmune, traumatic, endocrine medical conditions, or combinations thereof. In certain aspects, the medical condition can be an overactive bladder, fecal incontinence, urinary incontinence, other bladder and bowel disorders, and combinations thereof and the ultrasound can be applied through the sonolucent window to neuromodulate the sacral nerve/sacral root. Such conditions and nerves are only exemplary and other conditions and nerves can be targeted. The ultrasound energy can be delivered directionally (including bilaterally or unilaterally).

Referring to FIG. 10, in certain aspects, methods are provided herein that provide closed-loop therapy for a medical condition. For example, a method of improving a medical condition or a characteristic thereof in a patient 400 can comprise positioning a medical device with a sonolucent window at an internal body site associated with the medical condition such that the internal body site is accessible to ultrasound energy through the sonolucent window 402. Method 400 can comprise delivering therapy to the internal body site 404. The therapy can be, for example, electrical stimulation (including epidural electrical stimulation (EES)) or delivery of other energy forms such as ultrasound or acoustic, magnetic, optical energy. Alternatively or in addition, the therapy can comprise delivery of a chemical agent such as, for example, a pharmaceutical agent, a stem cell, small molecule, hormone, peptide, protein, antibiotic, or suitable combination thereof. Method 400 further can include applying, through the sonolucent window, ultrasound energy to the internal body site 406 and determining a physiological parameter associated with the medical condition based on the application of the ultrasound energy 408. Method 400 can further comprise adjusting the therapy based on the determination to improve the patient's medical condition or characteristic thereof. In the case of electrical stimulation, various parameters can be adjusted such as, for example, the pulse amplitude, pulse width, pulse frequency, duty cycle, waveform, or combinations thereof of an electric current. In the case of delivery of a chemical agent, the dosage or type of chemical agent can be adjusted, for example. Such parameters are only exemplary and other parameters can be adjusted to improve the patient's medical condition.

In certain aspects, the medical device is a bone scaffold that can be implanted in the void left in the vertebra(e) during decompression and stabilization surgery post-SCI. A laminectomy can be performed in the acute phase of SCI, which allows the decompression of the spinal cord at the level of the injury. The bone scaffold can be implanted such that it is over/above the spinal cord lesion. Once implanted, tsUS can be applied to the spinal cord lesion or a region adjacent thereto. For example, Doppler US imaging can be used to quantify blood flow values along the spinal cord blood vessels. In certain instances, the method of improving the SCI or characteristic thereof involves improving the patient's orthostatic hypotension and/or hemodynamic instability. Electrical stimulation, such as epidural electrical stimulation (EES), can be delivered below the spinal cord lesion and can modulate hemodynamic values such as blood flow in the spinal cord and improve orthostatic hypotension in patients within chronic SCI. EES, pharmaceutical or other chemical agents, or combinations thereof paired with a robust, time-resolved readout of spinal hemodynamics can provide a closed-loop therapy for spinal cord ischemia. To further enhance the tsUS, ultrasound microbubble contrast agents can be injected venously into the patient.

Such methods can facilitate safe and reproducible US imaging of the progression of spinal cord healing post-SCI decompression surgery; accelerate the development of bony tissue disrupted by the SCI; and/or facilitate adjusting interventional therapies to improve blood flow to the spinal cord lesion.

Each of the disclosed aspects and embodiments of the present disclosure may be considered individually or in combination with other aspects, embodiments, and variations of the disclosure. Further, while certain features of embodiments and aspects of the present disclosure may be shown in only certain figures or otherwise described in the certain parts of the disclosure, such features can be incorporated into other embodiments and aspects shown in other figures or other parts of the disclosure. Along the same lines, certain features of embodiments and aspects of the present disclosure that are shown in certain figures or otherwise described in certain parts of the disclosure can be optional or deleted from such embodiments and aspects. Additionally, when describing a range, all points within that range are included in this disclosure. Further, unless otherwise specified, none of the steps of the methods of the present disclosure are confined to any particular order of performance. Furthermore, all references cited herein are incorporated by reference in their entirety.

What is claimed is:

1. An implantable medical device configured to receive ultrasound (US) energy comprising:
an implantable device body having an outer surface, an inner surface, a first side, and a second side; and
a sonolucent window disposed in the device body and sized, configured, and fabricated from a sonolucent material to allow US energy to pass therethrough.

2. The implantable medical device of claim 1, wherein the sonolucent material has a mass density and an elastic modulus that corresponds to an intrinsic longitudinal sound speed value close to a nominal soft tissue sound speed of 1540 m/s and an acoustic impedance value close to a nominal soft tissue value of 1.5 MRayls.

3. The implantable medical device of claim 1, wherein the sonolucent material is homogenous and void free.

4. The medical device of claim 1, wherein the sonolucent window is removably disposed in the device body.

5. The medical device of claim 1, wherein the device body comprises a port sized and configured to receive a drug delivery needle.

6. The medical device of claim 1, wherein the sonolucent window comprises a plurality of sonolucent windows.

7. The medical device of claim 6, wherein each of the plurality of sonolucent windows are vertically adjacent to one another.

8. The medical device of claim 1, wherein the medical device body comprises a monolithic medical device body.

9. The medical device of claim 1, wherein the medical device body comprises a material that promotes bone regeneration, growth of new blood vessels, tissue repair, or combinations thereof.

10. The medical device of claim 1, wherein the device body comprises a polymeric material, a ceramic material, a composite material, a metallic material, or suitable combinations thereof.

11. A method of imaging an internal body site of a patient comprising:
implanting the medical device of claim 1 at the internal body site such that the internal body site is accessible to ultrasound energy through the sonolucent window; and
applying, through the sonolucent window, ultrasound energy to the internal body site to produce an image comprising the internal body site.

12. The method of claim 11, wherein the internal body site comprises the spinal cord.

13. A method of monitoring a medical condition or characteristic thereof in a patient comprising:
implanting the medical device of claim 1 at an internal body site associated with the medical condition such that the internal body site is accessible to ultrasound energy through the sonolucent window;
applying, through the sonolucent window, ultrasound energy to the internal body site;
measuring a physiological parameter associated with the medical condition based on the application of the ultrasound energy; and
monitoring the medical condition or characteristic thereof based on the measurement of the physiological parameter.

14. The method of claim 13, wherein the medical device is a bone scaffold; the medical condition is SCI, the characteristic is ischemia; the internal body site is a spinal cord lesion, the region adjacent to the spinal cord lesion, or both; and the physiological parameter is blood flow through or adjacent to the spinal cord lesion, the structure of the spinal cord at the spinal cord lesion or adjacent to the spinal cord lesion, or both.

15. The method of claim 13, wherein the medical condition is a myelopathy.

16. The method of claim 13, wherein measuring a physiological parameter associated with the medical condition based on the application of the ultrasound energy comprises quantifying blood flow values along the spinal cord vessels at the internal body site.

17. The method of claim 13, wherein the medical device is a bone scaffold; the medical condition is SCI, the characteristic is orthostatic, hypotension hemodynamic instability or both; the internal body site is a spinal cord lesion, the region adjacent to the spinal cord lesion, or both; and the physiological parameter is blood pressure.

18. A method of improving a medical condition or a characteristic thereof in a patient comprising:
implanting the medical device of claim 1 at an internal body site associated with the medical condition such that the internal body site is accessible to ultrasound energy through the sonolucent window;
delivering therapy to the internal body site;
applying, through the sonolucent window, ultrasound energy to the internal body site;
determining a physiological parameter associated with the medical condition based on the application of the ultrasound energy; and
adjusting the therapy based on the determination to improve the patient's medical condition or characteristic thereof.

19. The method of claim 18, wherein delivering therapy comprises delivery electrical stimulation, delivering a chemical agent, or both.

20. The method of claim 18, wherein the medical device is a bone scaffold; the medical condition is a spinal cord injury (SCI); the characteristic is orthostatic hypotension, hemodynamic instability or both; the internal body site is a spinal cord lesion, a region adjacent to the spinal cord lesion or both; and the physiological parameter is blood flow through or adjacent to the spinal cord lesion.

21. A method of improving a medical condition or characteristic thereof in a patient comprising:
implanting a medical device with a sonolucent window at an internal body site associated with the medical condition such that the internal body site is accessible to ultrasound energy through the sonolucent window;
applying, through the sonolucent window, ultrasound energy to the internal body site;
and improving the medical condition via application of the ultrasound energy.
